(19) United States
(12) Patent Application Publication
(10) Pub. No.: US 2020/0397691 A1
(43) Pub. Date: Dec. 24, 2020

(54) NICOTINE MOUTH SPRAY

(71) Applicant: Fertin Pharma A/S, Vejle (DK)

(72) Inventors: Bruno Provstgaard Nielsen, Vejle Øst (DK); Kent Albin Nielsen, Brande (DK)

(73) Assignee: Fertin Pharma A/S, Vejle (DK)

(21) Appl. No.: 16/770,313

(22) PCT Filed: Dec. 7, 2018
(86) PCT No.: PCT/DK2018/050337

§ 371 Date: Jun. 5, 2020

(30) Foreign Application Priority Data
Dec. 8, 2017 (DK) PA 2017 70927

(51) Int. Cl. A61K 9/00; A61K 9/08; A61K 31/465; A61P 25/34

(52) U.S. Cl. CPC A61K 9/006; A61P 25/34; A61K 31/465; A61K 9/08

(57) ABSTRACT

The invention relates to a liquid nicotine mouth spray formulation for oral mucosal delivery and fast onset nicotine craving relief, the mouth spray formulation being designed to adhere to the oral mucosa, and the mouth spray formulation being designed to utilize free nicotine base as both a source of nicotine and a source of pH regulating agent, wherein the mouth spray formulation does not contain any further pH regulating agent other than said free nicotine base.

### FIELD OF INVENTION

The invention relates to liquid nicotine mouth spray formulations, particularly to liquid nicotine mouth spray formulations without pH regulating agents other than nicotine and to liquid nicotine mouth spray formulations without any base regulating agent other than nicotine.

### BACKGROUND

Mouth spray is applied for the purpose of providing a release of nicotine in a user's mouth within a period of time.

A problem with the prior art is therefore that substantial amounts of the nicotine from prior art nicotine mouth spray may be swallowed by the user and thereby transferred to the stomach substantially without entering the bloodstream of the user.

A further problem may that how to buffer the pH value in the oral cavity to facilitate absorption of nicotine into the bloodstream, including adjusting the specific type of buffering agent and amount thereof to the specific formulation.

Accordingly, it is an object of the present invention to improve the utilization of the nicotine from the fast acting mouth spray by facilitating an improved actual uptake of nicotine through the mucous membrane of a user.

### SUMMARY

The invention relates to a liquid nicotine mouth spray formulation for oral mucosal delivery and fast onset nicotine craving relief, the mouth spray formulation being designed to adhere to the oral mucosa, and the mouth spray formulation being designed to utilize free nicotine base as both a source of nicotine and a source of pH regulating agent, wherein the mouth spray formulation does not contain any further pH regulating agent other than said free nicotine base.

Thus, when the mouth spray formulation being designed to utilize nicotine base as both a source of nicotine and a source of pH regulating agent for oral mucosal delivery, this means that the mouth spray formulation of the invention comprises nicotine base.

Also, since the mouth spray formulation does not contain any additional pH regulating agent other than nicotine base, no further pH regulating agent, such as e.g. buffering agents, are comprised in the mouth spray formulation other than nicotine.

Contrary to expectations, experiments have shown that the permeability of nicotine across the buccal mucosa decreases relatively little when increasing the concentration of nicotine. For example, experiments have shown that an increase in the concentration of nicotine from 100 microgram/mL to 14,000 microgram/mL results in a decrease of about a factor of two. This is highly surprising and is utilized by aiming for concentrations of nicotine in the oral cavity, which are much higher than previously seen or desired. The present delivery vehicle thus benefits and aims for very high nicotine content in the oral cavity, thereby increasing the nicotine uptake. Furthermore, it has been realized that the effect of nicotine concentrations is thus at least comparable to the effect of pH regulation in the oral cavity. This is contrary to any expectations.

One advantage of the invention may be that a fast onset of nicotine craving relief is obtained while at the same time minimizing burning in the throat. A significant challenge with oral administration of nicotine is that it often leads to a burning sensation in the throat. This burning sensation is normally worsened when increasing the release rate of nicotine. Thus, obtaining at the same time a fast onset of nicotine craving relief and also avoiding burning or minimizing burning in the throat is highly surprising.

In fact, a significant advantage of the invention may be that the fast onset nicotine craving relief may be obtained even without any additional pH regulating agent other than nicotine base, i.e. without any of the buffering agents that many conventional nicotine mouth spray includes.

Surprisingly, the fast onset of nicotine craving relief may be obtained without the usual assistance of pH regulating agents, and without causing burning. Usually, a fast onset of nicotine craving relief is assisted by pH regulating agents, such as buffering agents, for increasing the pH in order to facilitate efficient nicotine uptake. Without such pH regulating agents, an increase in nicotine concentration could help to support a high uptake of nicotine. However, a high nicotine concentration typically leads to burning sensation in the throat, which is high undesirable. Thus, it is very advantageous and surprising that the mouth spray formulation can attain a fast onset of nicotine craving relief without additional pH regulating agents, and reduced burning.

A further advantage of the invention may be that a desirable taste of the mouth spray may be obtained, compared to other products. Particularly, by avoiding the alkaline taste associated with many pH regulating agents and buffering agents, a mouth spray formulation having a more attractive taste for the user may be obtained.

A further advantage of the invention may be that chemical stability of the nicotine may be improved compared to mouth spray other products.

In embodiments of the invention, mouth spray formulation is designed to adhere to the oral mucosa by its composition having been adapted to adhere to the oral mucosa. This may in some embodiments be done by including a mucoadhesive.

In the present context, the mouth spray formulation is designed to utilize free nicotine base as both a source of nicotine and a source of pH regulating in the sense that the pH of the formulation is obtained by the use of nicotine without any further pH regulating agents.

In an embodiment of the invention, the formulation provides a peak saliva concentration of nicotine of more than 0.5 mg/mL and a peak saliva pH of more than 7.5 during the first 120 seconds upon oral administration.

In an embodiment of the invention, the formulation provides a peak saliva concentration of nicotine of more than 0.5 mg/mL and a peak saliva pH of more than 7.5 during the first 90 seconds upon oral administration.

In an embodiment of the invention, the formulation provides a peak saliva concentration of nicotine of more than 0.5 mg/mL and a peak saliva pH of more than 7.5 during the first 60 seconds upon oral administration.

In an embodiment of the invention, the formulation provides a peak saliva concentration of nicotine of more than 0.5 mg/mL during the first 90 seconds upon oral administration.

In an embodiment of the invention, the formulation provides a peak saliva concentration of nicotine of more than 0.5 mg/mL during the first 60 seconds upon oral administration.

In an embodiment of the invention, the formulation provides a peak saliva pH of more than 7.5 during the first 90 seconds upon oral administration.

In an embodiment of the invention, the formulation provides a peak saliva pH of more than 7.5 during the first 60 seconds upon oral administration.

In an embodiment of the invention, the formulation provides a peak saliva concentration of nicotine of more than 0.5 mg/mL and a peak saliva pH of more than 8 during the first 120 seconds upon oral administration.

In embodiments where the formulation provides a peak saliva concentration of nicotine of more than 0.5 mg/mL during the first 120 seconds upon oral administration, the amount of nicotine in the formulation should be adjusted to at least the amount necessary for obtaining this. Depending on the specific formulation, the amount of nicotine in the formulation may be higher than 0.5 mg in some embodiments, such as e.g. at least 1 mg or at least 2 mg.

In an embodiment of the invention, the mouth spray formulation comprises nicotine base in an amount of at least 0.5 mg.

The amount of nicotine content is generally given in amount per dosage unless otherwise specified. Since the dosage is referred to a mouth spray the amount will refer to the weight of the referred substance in the instructed dose, e.g. the amount of substance referred to in relation to a single spray or e.g. the amount of substance in the instructed number of sprays related to the instructed timing.

According to an embodiment of the invention the mouth spray formulation comprises nicotine base in an amount of between 0.5 and 4.0 mg.

According to an embodiment of the invention the mouth spray formulation comprises nicotine base in an amount of at least 0.5 mg per dosage.

In an embodiment of the invention, the mouth spray formulation has a pH of at least 7.5.

In an embodiment of the invention, the mouth spray formulation has a pH of at least 8.0.

According to an embodiment of the invention the mouth spray formulation has a pH of at least 8.5.

According to an embodiment of the invention the mouth spray formulation has a pH of at least 9.0.

According to an embodiment of the invention, the mouth spray formulation has a pH of 8.0 to 12.0.

In an embodiment of the invention, the mouth spray formulation has a pH of 9.0 to 10.0.

In an embodiment of the invention, the mouth spray formulation has a pH of 9.5 to 10.5.

In an embodiment of the invention, the mouth spray formulation comprises a mucoadhesive.

Thus, the mucoadhesive facilitates the adherence to the oral mucosa. I.e. in the above embodiment, the adherence provided by the mouth spray formulation being designed to adhere to the oral mucosa is facilitated or achieved by means said mucoadhesive.

In an embodiment of the invention, the mouth spray formulation comprises a mucoadhesive in the amount of between 1 and 50 mg/mL, such as in an amount of between 5 and 20 mg/mL.

In an embodiment of the invention, the mucoadhesive is selected from pectin, chitosan, alginate (e.g. sodium alginate), polyvinyl alcohol (PVA), polyacrylic acid (PAA), methyl cellulose (MC), sodium carboxy methylcellulose (SCMC), hydroxy propyl cellulose (HPC), preferably selected from the group consisting of pectin, PVA, PAA, xanthan gum, carbomer, carrageenan, and combinations thereof.

In an embodiment of the invention, the mucoadhesive comprises natural unbranched polysaccharides, such as alginate.

In an embodiment of the invention, the natural unbranched polysaccharides are adapted to form a bioadhesive gel upon administration to the oral cavity.

In an embodiment of the invention, the bioadhesive gel is formed by a cross-linking reaction with multivalent cations, such as multivalent cations in the oral cavity and/or multivalent cations release from the liquid nicotine mouth spray formulation.

In an embodiment of the invention, the liquid nicotine mouth spray formulation is designed for forming a gel after administering to the oral cavity.

In an embodiment of the invention, the liquid mouth spray formulation comprises nicotine in an amount of 1 mg/mL to 50 mg/mL, such as in an amount of 5 to 40 mg/mL.

In an embodiment of the invention, said nicotine is provided as a synthetic nicotine.

An advantage of the above embodiment may be that a more desirable taste profile may be obtained by avoiding undesirable taste notes that may be included in nicotine obtained from tobacco.

The invention further relates to a mouth spray formulation for oral mucosal delivery and for fast onset nicotine craving relief, the mouth spray formulation being designed to adhere to the oral mucosa, and the mouth spray formulation being designed to utilize free nicotine base as both a source of nicotine and a source of pH regulating agent, wherein pH in the mouth spray formulation is further regulated by an acid, and the mouth spray formulation does not contain any further pH base agent other than free nicotine base and the mouth spray formulation has a pH of at least 8.

In an embodiment of the invention, the acid is selected from pharmaceutically acceptable acids, such as hydrochloric acid.

In an embodiment of the invention, the liquid formulation comprises water.

Water may typically be included as a carrier or solvent. An especially advantageous embodiment is when the liquid formulation comprises water and the pH of the liquid formulation is at least 7.5.

In an embodiment of the invention, the liquid formulation comprises a pharmaceutically acceptable solvent selected from water; terpenes, such as menthol; alcohols, such as ethanol, propylene glycol, polyethylene glycol, such as PEG 400, glycerol and other similar alcohols; and mixtures or combinations thereof.

In an embodiment of the invention, the nicotine is not in ionic complex with a mucoadhesive water-soluble anionic polymer.

In an embodiment of the invention, the nicotine does not contain a nicotine complex.

The invention further relates to a liquid mouth spray formulation for oral mucosal delivery and for fast onset nicotine craving relief, the mouth spray formulation being designed to utilize free nicotine base as both a source of nicotine and a source of pH regulating agent, wherein pH in the mouth spray formulation is further regulated by an acid, and the mouth spray formulation does not contain any further pH base agent other than free nicotine base and the mouth spray formulation has a pH of at least 8.

According to an embodiment of the invention, the liquid mouth spray formulation of claim 19 is further limited by any of claims 1-18, with the provision that the limitations of claim 19 are adhered to.

The invention further relates to a liquid nicotine mouth spray formulation for use in fast onset nicotine craving relief, the mouth spray formulation being designed to adhere to the oral mucosa, and the mouth spray formulation being designed to utilize free nicotine base as both a source of nicotine and a source of pH regulating agent, wherein the mouth spray formulation does not contain any further pH regulating agent other than said free nicotine base and the mouth spray formulation has a pH of at least 8.

In the present context, it should be understood that said use in the alleviation of nicotine craving involves administering said liquid nicotine mouth spray formulation orally.

The invention further relates to a liquid nicotine mouth spray formulation for use in fast onset nicotine craving relief, the mouth spray formulation being designed to adhere to the oral mucosa, and the mouth spray formulation being designed to utilize free nicotine base as both a source of nicotine and a source of pH regulating agent, wherein pH in the mouth spray formulation is further regulated by an acid, and the mouth spray formulation does not contain any further pH base agent other than free nicotine base and the mouth spray formulation has a pH of at least 8.

In the present context, it should be understood that said use in the alleviation of nicotine craving involves administering said liquid nicotine mouth spray formulation orally.

The invention further relates to the liquid nicotine mouth spray formulation according to claim 17 or 18 and any of claims 1-16.

According to an embodiment of the invention, the liquid nicotine mouth spray formulation for oral mucosal delivery and fast onset nicotine craving relief is designed to adhere to the oral mucosa, and the mouth spray formulation is designed to utilize free nicotine base as both a source of nicotine and a source of pH regulating agent, wherein the mouth spray formulation does not contain any further pH regulating agent other than said free nicotine base, and wherein the mouth spray formulation comprises nicotine base in an amount of at least 0.5 mg.

According to an embodiment of the invention, the liquid nicotine mouth spray formulation for oral mucosal delivery and fast onset nicotine craving relief is designed to adhere to the oral mucosa, and the mouth spray formulation is designed to utilize free nicotine base as both a source of nicotine and a source of pH regulating agent, wherein the mouth spray formulation does not contain any further pH regulating agent other than said free nicotine base, and wherein the mouth spray formulation has a pH of at least 7.5.

According to an embodiment of the invention, the liquid nicotine mouth spray formulation for oral mucosal delivery and fast onset nicotine craving relief is designed to adhere to the oral mucosa, and the mouth spray formulation is designed to utilize free nicotine base as both a source of nicotine and a source of pH regulating agent, wherein the mouth spray formulation does not contain any further pH regulating agent other than said free nicotine base, wherein the mouth spray formulation comprises nicotine base in an amount of at least 0.5 mg, and wherein the mouth spray formulation has a pH of at least 7.5.

According to an embodiment of the invention, the liquid mouth spray formulation for oral mucosal delivery and for fast onset nicotine craving relief is designed to adhere to the oral mucosa, and the mouth spray formulation is designed to utilize free nicotine base as both a source of nicotine and a source of pH regulating agent, wherein pH in the mouth spray formulation is further regulated by an acid, and the mouth spray formulation does not contain any further pH base agent other than free nicotine base and the mouth spray formulation has a pH of at least 8, and wherein the mouth spray formulation comprises nicotine base in an amount of at least 0.5 mg.

According to an embodiment of the invention, the liquid mouth spray formulation for oral mucosal delivery and for fast onset nicotine craving relief is designed to adhere to the oral mucosa, and the mouth spray formulation is designed to utilize free nicotine base as both a source of nicotine and a source of pH regulating agent, wherein pH in the mouth spray formulation is further regulated by an acid, and the mouth spray formulation does not contain any further pH base agent other than free nicotine base and the mouth spray formulation has a pH of at least 8, and wherein the mouth spray formulation has a pH of at least 7.5.

According to an embodiment of the invention, the liquid mouth spray formulation for oral mucosal delivery and for fast onset nicotine craving relief is designed to adhere to the oral mucosa, and the mouth spray formulation is designed to utilize free nicotine base as both a source of nicotine and a source of pH regulating agent, wherein pH in the mouth spray formulation is further regulated by an acid, and the mouth spray formulation does not contain any further pH base agent other than free nicotine base and the mouth spray formulation has a pH of at least 8, wherein the mouth spray formulation comprises nicotine base in an amount of at least 0.5 mg, and wherein the mouth spray formulation has a pH of at least 7.5.

In an embodiment of the invention, the liquid mouth spray formulation of claim 11 may be combined with the liquid mouth spray of any of claims 2-10, with the reservation that the limitations of claim 1 are not adhered to.

The invention further relates to a method of alleviation of nicotine craving relief by administering an effective amount of said oral nicotine formulation according to any of its embodiments.

In an embodiment of the invention, the method comprises the step of administering the formulation to a local area of the buccal cavity.

In an embodiment of the invention, the method according to any of its embodiments comprises the step of administering the formulation to a local area of the upper buccal cavity.

In an embodiment of the invention the method comprises the step of administering the formulation is administered under the tongue, i.e. sublingually.

This is even more advantageous, given the fact that very high concentrations of nicotine may be obtained sublingually with only minimum burning in the throat. A very high sublingually uptake thus both keeps the burning at a minimum and increases the nicotine uptake at the same time.

Moreover, the liquid nicotine mouth spray formulation according to the invention or any of its embodiments for use in the method of the invention or any of its embodiments.

The invention further relates to a method a nicotine mouth spray device comprising the liquid nicotine mouth spray formulation of the invention or any of its embodiments.

In an embodiment of the invention, the nicotine mouth spray device is configured to administer a predefined amount of the nicotine mouth spray formulation for each administration.

In an embodiment of the invention, the nicotine mouth spray device is configured to administer a predefined amount of 0.01 to 1.0 mL of the nicotine mouth spray formulation for each administration.

### DETAILED DESCRIPTION

As used herein the term “liquid mouth spray formulation” refers to a mouth spray for application of drug orally, e.g. either sublingually or buccal. The mouth spray formulation is provided as a liquid, but may comprise gelling agents for forming a gel during/after administering to the oral cavity. Liquid mouth spray formulation may also be referred to as fast acting mouth spray.

As used herein, the term “dissolve” refer to the process of a liquid formulation being mixed with and thus dissolved in the saliva. Unless otherwise stated, dissolving implies a full dissolving of the compound in question.

As used herein, the term “mouth spray” refers to a small pump-type or squeeze-type container having a spray nozzle and contains a liquid (mouth spray) to be sprayed into the mouth.

As used herein, the term “nicotine” refers to nicotine in any form, including free base nicotine, nicotine salts, nicotine bound to ion exchange resins, such as nicotine polacrilex, nicotine bound to zeolites; nicotine bound to cellulose, such as microcrystalline cellulose, such as of microbial origin, or starch microspheres, and mixtures thereof. Thus, when referring to nicotine amounts, the amounts refers to the amount of pure nicotine. Thus, when measuring the concentration of nicotine added as nicotine salt, it is the mass of the equivalent amount of pure nicotine, not the mass of the salt, that is relevant. Nicotine also covers nicotine not obtained from tobacco, often referred to as synthetic nicotine.

As used herein, the term “nicotine salt” refers to nicotine in ionized form bonded electrostatically to a counterion.

As used herein, the term “NBT” refers to nicotine bitartrate and hydrates thereof.

As used herein, the term “%” and “percent” refers to percent by weight, unless otherwise is stated.

As used herein, the term “release of nicotine” refers to the nicotine being made bioavailable, i.e. available for absorption over the mucous membrane in the oral cavity. While some forms of nicotine require dissolution for being bioavailable, other forms may be readily absorbed into the body without dissolution.

As used herein, the term “peak saliva concentration of nicotine” refers to the peak value of the concentration of nicotine in saliva of the oral cavity, where the saliva includes delivery vehicle of the nicotine dissolved therein, e.g. liquid mouth spray formulation dissolved in the saliva. Also, it should be understood that the peak saliva concentration is considered to be achieved whenever the criterion is fulfilled. E.g. if a peak saliva concentration of nicotine is at least 0.5 mg/mL, this peak saliva concentration is achieved whenever the concentration of nicotine exceeds 0.5 mg/mL. Measurements of peak saliva nicotine concentration is performed as follows:

One dosage of the formulation is administered sublingually to at least six individuals. At specified time intervals, the saliva is collected. The experiment is repeated. Thus, each nicotine concentration value is the arithmetic mean of 12 measurements, i.e. performed on saliva-samples from six individuals times 2. The nicotine concentration of saliva is analyzed on HPLC after extraction into relevant buffer.

As used herein, the term “peak saliva pH” refers to the peak value of the pH in saliva of the oral cavity, where the saliva includes any delivery vehicle of the pH regulating agent, such as e.g. liquid mouth spray formulations etc. Also, it should be understood that the peak saliva pH is considered to be achieved whenever the criterion is fulfilled. E.g. if a peak saliva pH is at least 7.5, this peak saliva pH is achieved whenever the pH exceeds 7.5. Peak saliva pH is measured in vivo and is measured as follows:

At least 6 individuals chewed on a gum base free of buffer for 1 minute, after which the initial pH in a sample from the saliva from each of the individuals is measured with a suitable pH-electrode system, e.g. a stainless steel electrode PHW77-SS. Only individuals having, after chewing on a gum base free of buffer for one minute, an initial pH in the saliva inside the range from 6.7 and 7.3 are selected. These individuals thereby qualify as average individuals.

One dosage of the formulation is administered sublingually to at least six individuals. Hereafter, the saliva pH from each of the six individuals is measured at specified time intervals. Thus, each pH-value is the arithmetic mean of six measurements performed on saliva-samples from six individuals.

As used herein, the term “pH regulating agent” refers to agents, which active adjust and regulates the pH value of the solution to which they have been added or are to be added, including buffering agents. Thus, as used herein pH regulating agents are strong acids (i.e. acids that are completely dissociated in aqueous solution) and strong bases (i.e. bases that are completely dissociated in aqueous solution), acidic buffering agents and alkaline buffering agents, and nicotine. On the other hand, pH regulating agents does not including substances and compositions that can only affect the pH by dilution. Furthermore, pH regulating agents does not include e.g. flavoring, fillers, etc. Within the present invention, nicotine is considered a pH regulating agent. In embodiments of the invention, pH regulating agents include acids and bases, including acidic buffering agents and alkaline buffering agents.

As used herein, the term “buffering agent” is used interchangeably with “buffer” and refers to agents for obtaining a buffer solution. Buffering agents include acidic buffering agents, i.e. for obtaining a buffer solution with an acidic pH, and alkaline buffering agents, i.e. for obtaining a buffer solution with an alkaline pH.

As used herein, the term “fast onset nicotine craving relief” refers to relief of nicotine craving, for which the onset is relatively fast, i.e. only a relatively short period of time after oral administering. In embodiments of the invention, the fast onset refers to a period after oral administration until craving relief is experienced being no more than 180 seconds, such as no more than 120 seconds, such as no more than 60 seconds.

### EXAMPLES

The following non-limiting examples illustrate different variations of the present invention.

### Example 1

### Preparation of Fast Acting Mouth Spray

In the present example six fast acting mouth spray are prepared with formulations as outlined in table 1. Four of the fast acting mouth spray are prepared with pure nicotine base and two is placebo. The three first batches contain no buffer whereas the last three batches contains buffer. Some of the six batches are adjusted with pH regulating agents for obtaining pH 9.0 of the final mixture. See further explanation in table 2.

TABLE 1
High level description of Fast acting mouth spray compositions.

| | Description of trial |
|---|---|
| FAM(a) | No buffer system - Placebo trial |
| FAM(b) | No buffer system - pH adjusted to 9.0 |
| FAM(c) | No buffer system - not adjusted |
| FAM(d) | Buffer system - Placebo trial |
| FAM(e) | Buffer system pH - adjusted to 9.0 |
| FAM(f) | Buffer system - not adjusted |

TABLE 2
Fast acting mouth spray compositions. Amounts are
given in percent by weight of each composition.

| | FAM | FAM | FAM | FAM | FAM | FAM |
|---|---|---|---|---|---|---|
| | (a) | (b) | (c) | (d) | (e) | (f) |
| Nicotine base | N/A | 1.43 | 1.43 | N/A | 1.43 | 1.43 |
| Dem. water | 60.9 | 59.47 | 59.47 | 58.5 | 57.07 | 57.07 |
| Poloxamer 407 | 3.0 | 3.0 | 3.0 | 3.0 | 3.0 | 3.0 |
| Propylene | 12.5 | 12.5 | 12.5 | 12.5 | 12.5 | 12.5 |
| glycol | | | | | | |
| Glycerine | 12.5 | 12.5 | 12.5 | 12.5 | 12.5 | 12.5 |
| Peppermint | 0.30 | 0.30 | 0.30 | 0.30 | 0.30 | 0.30 |
| Menthol | 0.50 | 0.50 | 0.50 | 0.50 | 0.50 | 0.50 |
| Acesulfame K | 0.20 | 0.20 | 0.20 | 0.20 | 0.20 | 0.20 |
| Sucralose | 0.1 | 0.1 | 0.1 | 0.1 | 0.1 | 0.1 |
| Sodium | — | — | — | 1.2 | 1.2 | 1.2 |
| carbonate | | | | | | |
| Trometamol | — | — | — | 1.2 | 1.2 | 1.2 |
| Ethanol | 10.0 | 10.0 | 10.0 | 10.0 | 10.0 | 10.0 |
| Total | 100.0 | 100.0 | 100.0 | 100.0 | 100.0 | 100.0 |

FAM = Fast acting mouth spray.

The fast acting mouth spray are manufactured on a lab scale using a bench scale magnetic stirrer. The assay, in vitro pH and viscosity are measured after manufacture to ensure they match the acceptance criteria.

Raw materials are weighed from bags or containers into separate weighing containers except for demineralized water. The batch size is 210 grams.

### Preparing the Mixture:

Demineralized water of room temperature is added to a blue cap bottle (size 2× expected batch volume). Add a stir bar (magnet) and place the glass bottle on a magnetic stirrer. No heating is needed. Add surfactant (for example Poloxamer 407) slowly to the water while stirring. Stir until it is dissolved. Add all other excipients for and stir until fully dissolved.

Nicotine base is added using a 3.0 ml glass pipette, and the liquid is stirred for at least 5 minutes with stirring showing visible vortex. The pH of the solution is measured. The pH of the final mixture is checked and where applicable adjusted to pH 9.0 with 2 M HCl or 2 M NaOH. The liquid is stirred during addition and the mixture is stirred for 5 minutes. The pH of the final mixture is measured and results are shown in table 3.

TABLE 3
pH in final mixture of FAM(a-f)

| | Mixture pH | | |
|---|---|---|---|
| | Before | | |
| | addition of | After | |
| | nicotine | addition of | Adjustment |
| | base | nicotine base | of pH |
| FAM(a) | 6.73 | — | — |
| FAM(b) | — | 9.85 | 9.01 |
| FAM(c) | — | 9.95 | — |
| FAM(d) | 9.32 | — | — |
| FAM(e) | — | 9.41 | 9.01 |
| FAM(f) | — | 9.42 | — |

The liquid is filled into HDPE or PET bottles. The filling volume is checked by weight. The bottle is closed with a pump spray head with an output volume of 70 microliters in this case corresponding to a final dose of 1 mg nicotine due to the nicotine concentration of the liquid being 14.3 mg/ml. The output volume could be adjusted from 50 to 150 microliters with or without changing the nicotine concentration of the liquid.

The fast acting mouth spray according to the invention may comprise coloring agents. According to an embodiment of the invention, the fast acting mouth sprays may comprise color agents and whiteners such as FD&C-type dyes and lakes, fruit and vegetable extracts, and combinations thereof.

### Example 2

Preparation of Fast Acting Mouth Spray with Different Concentrations

In the present example six fast acting mouth sprays are prepared with formulations as outlined in table 4A. The fast acting mouth spray is prepared with nicotine pure base. The methodology for manufacture is similar to the description in example 1.

TABLE 4A
Fast acting mouth spray compositions. Amounts are
given in percent by weight of each composition.

| | FAM | FAM | FAM | FAM | FAM | FAM |
|---|---|---|---|---|---|---|
| | (g) | (h) | (i) | (j) | (k) | (l) |
| Nicotine base | 0.72 | 1.43 | 2.86 | 0.72 | 1.43 | 2.86 |
| Dem. water | 60.18 | 59.47 | 58.04 | 58.38 | 57.67 | 56.24 |
| Poloxamer 407 | 3.0 | 3.0 | 3.0 | 3.0 | 3.0 | 3.0 |
| Propylene | 12.5 | 12.5 | 12.5 | 12.5 | 12.5 | 12.5 |
| glycol | | | | | | |
| Glycerine | 12.5 | 12.5 | 12.5 | 12.5 | 12.5 | 12.5 |
| Peppermint | 0.3 | 0.3 | 0.3 | 0.3 | 0.3 | 0.3 |
| Menthol | 0.1 | 0.1 | 0.1 | 0.1 | 0.1 | 0.1 |
| Acesulfame K | 0.2 | 0.2 | 0.2 | 0.2 | 0.2 | 0.2 |
| Sucralose | 0.1 | 0.1 | 0.1 | 0.1 | 0.1 | 0.1 |
| Trometamol | — | — | — | 1.35 | 1.35 | 1.35 |
| Sodium | — | — | — | 0.45 | 0.45 | 0.45 |
| bicarbonate | | | | | | |
| Ethanol 96% | 10.4 | 10.4 | 10.4 | 10.4 | 10.4 | 10.4 |
| Total | 100.0 | 100.0 | 100.0 | 100.0 | 100.0 | 100.0 |

FAM = Fast acting mouth spray.

Further, three additional fast acting mouth sprays comprising mucoadhesive are prepared with formulations as outlined in table 4A. The fast acting mouth spray is prepared with pure, free nicotine base. The methodology for manufacture is similar to the description in example 1.

TABLE 4B
Fast acting mouth spray compositions. Amounts are
given in percent by weight of each composition.

| | FAM (m) | FAM (n) | FAM (o) |
|---|---|---|---|
| Nicotine base | 0.72 | 1.43 | 2.86 |
| Dem. water | 59.18 | 58.47 | 57.04 |
| Poloxamer 407 | 3.0 | 3.0 | 3.0 |
| Propylene glycol | 12.5 | 12.5 | 12.5 |
| Glycerine | 12.5 | 12.5 | 12.5 |
| Peppermint | 0.3 | 0.3 | 0.3 |
| Menthol | 0.1 | 0.1 | 0.1 |
| Acesulfame K | 0.2 | 0.2 | 0.2 |
| Sucralose | 0.1 | 0.1 | 0.1 |
| Sodium alginate | 1.0 | 1.0 | 1.0 |
| Ethanol 96% | 10.4 | 10.4 | 10.4 |
| Total | 100.0 | 100.0 | 100.0 |

FAM = Fast acting mouth spray.

As can be seen in table 2, demineralized water, propylene glycol, glycerine, and ethanol 96% are used as pharmaceutically acceptable solvents. As can be seen in table 4A-4B, demineralized water, propylene glycol, and glycerine are used as pharmaceutically acceptable solvents. Examples of usable pharmaceutically acceptable solvents include water; terpenes, such as menthol; alcohols, such as ethanol, propylene glycol, polyethylene glycol, such as PEG 400, glycerol and other similar alcohols; and mixtures or combinations thereof.

In an embodiment of the invention, the pharmaceutically acceptable solvents comprise propylene glycol.

In an embodiment of the invention, the pharmaceutically acceptable solvents comprise PEG 400.

In an embodiment of the invention, the pharmaceutically acceptable solvents comprise glycerol.

In an embodiment of the invention, the pharmaceutically acceptable solvents comprise ethanol.

In an embodiment of the invention, the pharmaceutically acceptable solvents comprise water.

In an embodiment of the invention, said liquid formulation comprises glycerol in an amount of 0-40% by weight, such as 0.01-40% by weight, such as 0.1-40% by weight.

In an embodiment of the invention, said liquid formulation comprises propylene glycol in an amount of 0-40 by weight, such as 0.01-40% by weight, such as 0.1-40% by weight.

In an embodiment of the invention, said liquid formulation comprises 0.1-70% by weight of water, such as 0.1-60% by weight of water, such as 0-10% by weight of water, or such as 30-50% by weight of water.

In an embodiment of the invention, said liquid formulation comprises water in an amount of 20-80% by weight of the liquid formulation, such as 30-75% by weight of the liquid formulation, such as 40-70% by weight of the liquid formulation.

As can be seen in table 4A-4B, peppermint and menthol are used as flavors. Usable flavors include almond, almond amaretto, apple, Bavarian cream, black cherry, black sesame seed, blueberry, brown sugar, bubblegum, butterscotch, cappuccino, caramel, caramel cappuccino, cheesecake (graham crust), cinnamon redhots, cotton candy, circus cotton candy, clove, coconut, coffee, clear coffee, double chocolate, energy cow, graham cracker, grape juice, green apple, Hawaiian punch, honey, Jamaican rum, Kentucky bourbon, kiwi, koolada, lemon, lemon lime, tobacco, maple syrup, maraschino cherry, marshmallow, menthol, milk chocolate, mocha, Mountain Dew, peanut butter, pecan, peppermint, raspberry, banana, ripe banana, root beer, RY 4, spearmint, strawberry, sweet cream, sweet tarts, sweetener, toasted almond, tobacco, tobacco blend, vanilla bean ice cream, vanilla cupcake, vanilla swirl, vanillin, waffle, Belgian waffle, watermelon, whipped cream, white chocolate, wintergreen, amaretto, banana cream, black walnut, blackberry, butter, butter rum, cherry, chocolate hazelnut, cinnamon roll, cola, creme de menthe, eggnog, English toffee, guava, lemonade, licorice, maple, mint chocolate chip, orange cream, peach, pina colada, pineapple, plum, pomegranate, pralines and cream, red licorice, salt water taffy, strawberry banana, strawberry kiwi, tropical punch, tutti frutti, vanilla, or any combination thereof.

According to an advantageous embodiment of the invention, said liquid formulation comprises 0.01-5% by weight of flavoring, such as 0.01-2.5% by weight of flavoring, 0.01-0.5% by weight of flavoring.

According to an embodiment of the invention, flavor may be used as taste masking for the nicotine.

In embodiments of the invention, the formulation comprises pH regulating agent in an amount of from 0.5% to 5.0% by weight of the formulation.

As can be seen in table 2, acesulfame K and sucralose are used as high intensity sweeteners. Usable high intensity sweeteners include, but are not limited to sucralose, aspartame, salts of acesulfame, such as acesulfame potassium, alitame, saccharin and its salts, cyclamic acid and its salts, glycyrrhizin, dihydrochalcones, thaumatin, monellin, stevioside and the like, alone or in combination.

In embodiments of the invention, the liquid formulation comprise one or more fast acting mouth spray ingredients selected from the group consisting solvents, flavors, surfactants, emulsifiers, antioxidants, enhancers, carriers, absorption enhancers, high intensity sweeteners, mucoadhesives, colors, or any combination thereof.

As can be seen in table 2, poloxamer 407 is used as a surfactant. Other surfactants may also be used in some embodiments.

Usable emulsifiers include, but are not limited to, the emulsifiers are selected from the group consisting of glyceryl monostearate, propylene glycol monostearate, mono- and diglycerides of edible fatty acids, lactic acid esters and acetic acid esters of mono- and diglycerides of edible fatty acids, acetylated mono and diglycerides, sugar esters of edible fatty acids, Na-, K-, Mg- and Ca-stearates, poloxamer 407, lecithin, hydroxylated lecithin and combinations thereof.

In an embodiment of the invention, the mucoadhesive is selected from pectin, chitosan, alginate (e.g. sodium alginate), polyvinyl alcohol (PVA), polyacrylic acid (PAA), methyl cellulose (MC), sodium carboxy methylcellulose (SCMC), hydroxy propyl cellulose (HPC), preferably selected from the group consisting of pectin, PVA, PAA, xanthan gum, carbomer, carrageenan, and combinations thereof.

### Example 3

### In Vivo pH Profile

Table 5 shows the pH profiles over time for a number of fast acting mouth spray as well as for a commercially available mouth spray. The nicotine mouth spray reveals also fast craving relief.

TABLE 5
pH In vivo measurements.

| Time (min) | Pretest | 0 | 1 | 3 |
|---|---|---|---|---|
| Commercial | 7.1 | 8.3 | 7.5 | 7.2 |
| mouth spray | | | | |
| FAM(a) | 6.9 | 7.1 | 7.1 | 7.0 |
| FAM(b) | 7.0 | 7.8 | 7.3 | 7.1 |
| FAM(c) | 7.0 | 7.7 | 7.3 | 7.2 |
| FAM(d) | 7.0 | 8.1 | 7.3 | 7.1 |
| FAM(e) | 7.0 | 8.2 | 7.3 | 7.1 |
| FAM(f) | 7.1 | 8.1 | 7.4 | 7.2 |

The measurements of the average in vivo pH values given in Table 5 were performed as follows:

At least 6 individuals chewed on a gum base free of buffer for 1 minute, after which the initial pH in a sample from the saliva from each of the individuals was measured with a suitable pH-electrode system, e.g. a stainless steel electrode PHW77-SS. None of the individuals had, after chewing on a gum base free of buffer for one minute, an initial pH in the saliva outside the range from 6.7 and 7.3. The individuals thereby qualified as average individuals.

Then the six individuals applied one dose of the fast acting mouth spray sublingually. Hereafter the saliva pH from each of the six individuals was measured at specified time intervals. Thus, each pH-value in Table 5 is the arithmetic mean of six measurements performed on saliva-samples from six individuals. The sample volume of the individual saliva-samples may vary because the volume of saliva obtained may be different from each individual. This difference in sample volume does not affect the pH-measurements significantly. Also, it has been established by appropriate tests that a variation in time between collections of samples does not significantly alter the result. This means that the measured pH-value after three minutes is not significantly affected by whether another saliva-sample is taken from the six individuals e.g. after two minutes or not. Furthermore, it has been established by appropriate tests that the time from taking a sample to the time of measuring is not critical to the measured value. However, in the present measurements, the pH-values were measured in the samples within at most 15 minutes of sample collection.

It should be noted that the in vivo pH-profile is different from an in vitro pH-profile due to the fact that acidic sodium bicarbonate is normally continuously produced in saliva, hence neutralizing the alkaline contribution from buffer. Thus, the pH obtained in vivo will be lower than in vitro measured in a beaker with stirring.

### Example 4

### Nicotine Concentration in Saliva

The measurements of the average nicotine concentration in saliva were performed as follows:

At least six individuals applied one dose of the fast acting mouth spray sublingually given in example 2. After 30 seconds, the saliva was collected. The experiment was repeated. Thus, each nicotine concentration value is the arithmetic mean of 12 measurements, i.e. performed on saliva-samples from six individuals times 2. The nicotine concentration of saliva was analyzed on HPLC after extraction into relevant buffer. Furthermore, compared to a commercially available mouth spray.

It is seen that the release of nicotine may vary a lot between the disclosed fast acting mouth spray. Hereby a release profile as desired may be used together with a high pH (as seen in example 3), whereby the nicotine may be more efficiently used.

Obtained in vivo saliva concentrations of nicotine are outlined in table 6.

TABLE 6
Nicotine concentration in saliva after 1 spray dose for mouthsprays
FAM(h), FAM(i), FAM(k), FAM(l) and Nicorette Quickmist.

| | | | | | Nicorette |
|---|---|---|---|---|---|
| | FAM(h) | FAM(i) | FAM(k) | FAM(l) | Quickmist |
| Nicotine per | 1 mg | 2 mg | 1 mg | 2 mg | 1 mg |
| spray dose | | | | | |
| Nicotine | 0.51 | 1.03 | 0.49 | 0.95 | 0.40 |
| concentration | | | | | |
| [mg/mL] | | | | | |

As can be seen from table 6, a nicotine concentration of about 1 mg/mL is obtained by FAM(i) without using buffer. FAM(l), including buffer, results in a similar nicotine concentration. The same trend is observed when comparing FAM(h) without buffer and FAM(k) with buffer. Thus, the liquid mouthspray formulations of the invention are desirable for obtaining a peak saliva nicotine concentration of more than 0.5 mg/mL. The obtained in vivo saliva nicotine concentrations were slightly higher than for the commercial mouthspray having corresponding nicotine dose per spray.

### Example 5

### Evaluation of Fast Acting Mouth Spray—Burning

In general experiments have disclosed that nicotine fast acting mouth spray according to the invention result in high absorption efficiency of nicotine into the blood stream for a nicotine fast acting mouth spray. With such fast integration, high pH-value combined with high nicotine concentration, a minor part of the nicotine is swallowed by the user instead of entering the blood system resulting in fast craving relief.

When pH in the mouth is high, the nicotine is used in a very efficient way. However, too high pH in the saliva of the fast acting mouth spray users may not be desirable, since the highly alkaline pH-value results in problems with irritation and burning of the sublingual tissue.

Consequently, the fast acting mouth spray of the invention are indeed suitable in that they provide an efficient utilization of nicotine and at the same time are pleasant to the user, i.e. with clearly diminished unwanted side effects, hereunder particularly burning in the throat.

Burning in the throat was evaluated for FAM(h) and Nicorette Quickmist. A predetermined dose corresponding to 1 mg nicotine is administered to the oral cavity as indicated in table 7. Evaluation of burning sensation is performed as described in the following.

Burning in the throat was evaluated by a test panel of 5 trained individuals. Each individual evaluates the burning from 1 to 15, were 15 is the most intense burning. The evaluations are noted for the time periods indicated. Average values are calculated and are indicated in table 7.

TABLE 7
Sensory evaluation of throat burning

Time [seconds]

| | | | | | |
|---|---|---|---|---|---|
| 25 | 55 | 85 | 120 | 145 | 175 |

Burning score (1-15)

| | | | | | | |
|---|---|---|---|---|---|---|
| FAM(h) | 0.92 | 3.25 | 3.85 | 3.75 | 3.74 | 3.57 |
| Nicorette | 1.53 | 6.55 | 6.67 | 6.58 | 6.21 | 5.92 |
| Quickmist | | | | | | |

As can be seen from table 7, the mouthspray FAM(h) of the invention gives significantly lower burning than the comparison mouthspray. Thus, the liquid mouthspray formulations of the invention supports obtaining a low throat burning sensation.

### Example 6

### Nicotine Absorption

Nicotine absorption was tested in vivo for FAM(h), FAM(i), FAM(k), FAM(l) and commercially available Nicorette Quickmist. A predefined spray dose of 70 microliters corresponding to 1 or 2 mg nicotine was administered to the oral cavity, as outlined in table 8.

No swallowing was allowed within the first 30 seconds. The saliva is collected after 30 seconds in 50 mL centrifugal tubes. These are analysed to determine the content of nicotine. The absorption is estimated as the difference between initial dose and the content of nicotine in saliva.

The results are shown in table 8.

TABLE 8
Nicotine absorption.

| | Nicotine | | | |
|---|---|---|---|---|
| | concentration | | | |
| | in spray | Dose | | % wt. |
| Batch no. | [mg/g] | [mg] | Buffer | absorbed |
| FAM(h) | 14.3 | 1.0 | No buffer | 51 |
| FAM(i) | 28.6 | 2.0 | No buffer | 48 |
| FAM(k) | 14.3 | 1.0 | Buffer | 53 |
| FAM(l) | 28.6 | 2.0 | Buffer | 53 |
| Nicorette | 14.3 | 1.0 | Buffer: | 60 |
| QuickMist | | | Trometamol, | |
| | | | Sodium | |
| | | | hydrogen | |
| | | | carbonate | |

It is noted that nicotine absorption was above 40% by weight, and for FAM(h) even above 50% by weight. Also, when comparing the mouthsprays FAM(h)-FAM(i) with corresponding mouthsprays comprising buffer, FAM(k)-FAM(l), the nicotine absorption of FAM(h)-FAM(i) is only slightly below that of FAM(k)-FAM(l), which is contrary to expectations. Hence, it appears that similar levels of absorption may be achieved with mouthsprays according to the invention as compared to mouthsprays containing buffer.

These very high results for nicotine absorption of buffer free mouthsprays, approximately at the level of buffer-containing mouthsprays, ensures that effective alleviation of nicotine craving relief is obtained by administration of the inventive, liquid mouthspray formulation to the oral cavity.

1. A liquid nicotine mouth spray formulation for oral mucosal delivery and fast onset nicotine craving relief, the mouth spray formulation being designed to adhere to the oral mucosa, and the mouth spray formulation being designed to utilize free nicotine base as both a source of nicotine and a source of pH regulating agent, wherein the mouth spray formulation does not contain any further pH regulating agent other than said free nicotine base.

2. The liquid nicotine mouth spray formulation according to claim 1, wherein the formulation provides a peak saliva concentration of nicotine of more than 0.5 mg/mL and a peak saliva pH of more than 7.5 during the first 120 seconds upon oral administration.

3. The liquid nicotine mouth spray formulation according to claim 1, wherein the mouth spray formulation comprises nicotine base in an amount of at least 0.5 mg.

4. (canceled)

5. The liquid nicotine mouth spray formulation according to claim 1, wherein the mouth spray formulation has a pH of at least 8.0.

6. The liquid nicotine mouth spray formulation according to claim 1, wherein the mouth spray formulation has a pH of 9.0 to 10.0.

7. The liquid nicotine mouth spray formulation according to claim 1, wherein the mouth spray formulation has a pH of 9.5 to 10.5.

8. The liquid nicotine mouth spray formulation according to claim 1, wherein the mouth spray formulation comprises a mucoadhesive.

9. The liquid nicotine mouth spray formulation according to claim 8, wherein the mucoadhesive is selected from pectin, chitosan, alginate, polyvinyl alcohol (PVA), polyacrylic acid (PAA), methyl cellulose (MC), sodium carboxy methylcellulose (SCMC), hydroxy propyl cellulose (HPC), xanthan gum, carbomer, carrageenan, and combinations thereof.

10. The liquid nicotine mouth spray formulation according to claim 1, wherein the mouth spray formulation comprises a mucoadhesive in the amount of between 1 and 50 mg/mL.

11. The liquid nicotine mouth spray formulation according to claim 8, wherein the liquid nicotine mouth spray formulation is designed for forming a gel after administering to the oral cavity.

12. The liquid nicotine mouth spray formulation according to claim 1, wherein the liquid mouth spray formulation comprises nicotine in an amount of 1 mg/mL to 50 mg/mL.

13. The liquid nicotine mouth spray formulation according to claim 1, wherein said nicotine is provided as a synthetic nicotine.

14-16. (canceled)

17. The liquid nicotine mouth spray formulation according to claim 1, wherein the liquid formulation comprises a pharmaceutically acceptable solvent selected from water; terpenes; alcohols and mixtures or combinations thereof.

18-20. (canceled)

21. A liquid nicotine mouth spray formulation for use in fast onset nicotine craving relief, the mouth spray formulation being designed to adhere to the oral mucosa, and the mouth spray formulation being designed to utilize free nicotine base as both a source of nicotine and a source of pH regulating agent, wherein the mouth spray formulation does not contain any further pH regulating agent other than said free nicotine base and the mouth spray formulation has a pH of at least 8.

22-23. (canceled)

24. A method of alleviation of nicotine craving relief by administering an effective amount of said oral nicotine formulation according to claim 1.

25. The method according to claim 24, wherein the method comprises the step of administering the formulation to a local area of the buccal cavity.

26. The method according to claim 15, wherein the method comprises the step of administering the formulation to a local area of the upper buccal cavity.

27. The method according to claim 15, wherein the method comprises the step of administering the formulation is administered under the tongue.

28. Use of the liquid nicotine mouth spray formulation according to claim 1 in alleviation of nicotine craving relief by administering an effective amount of said oral nicotine formulation.

29. A nicotine mouth spray device comprising the liquid nicotine mouth spray formulation according to claim 1.
